(19) United States
(12) Patent Application Publication
(10) Pub. No.: US 2006/0052457 A1
(43) Pub. Date: Mar. 9, 2006

(54) Use of ppar alpha agonists for the treatment of vascular and renal diseases

(76) Inventors: Peter Zahradka, Manitoba (CA); Carla Taylor, Manitoba (CA)

Correspondence Address: Ade & Company, 1700-360 Main Street, Winnipeg Manitoba, R3C 3Z3, CA

(21) Appl. No.: 10/507,495

(22) PCT Filed: Mar. 11, 2003
(86) PCT No.: PCT/CA03/00335

§ 371 Date: Aug. 17, 2005

Related U.S. Application Data
(60) Provisional application No. 60/362,243, filed on Mar. 11, 2002.

(51) Int. Cl. A61K 31/192

(52) U.S. Cl. 514/571

(57) ABSTRACT

Activation of peroxisome proliferator activated receptor alpha (PPARα) by administration of therapeutic amounts of a PPARα agonist, WY-14643, inhibits the proliferation of vascular smooth muscle cells, hepatoma cells and human renal proximal tubule cells. WY-14643 may be applicable as a medicament for the treatment of proliferative vascular disease (atherosclerosis, hypertension), revascularization-induced injury (restenosis) and chronic renal failure.

This application is derived from Provisional Patent Application Ser. No. 60/362,243, filed Mar. 11, 2002.

### FIELD OF THE INVENTION

The present invention relates generally to the field of pharmaceutical compositions and disease treatments.

### BACKGROUND OF THE INVENTION

The peroxisome proliferator-activated receptors (PPARs) belong to the nuclear receptor superfamily of ligand-activated transcription factors that include the steroid, retinoid and thyroid hormone receptors (Torra et al., 2001, Curr Opin Lipidol 12: 245-254; Qi et al., 2000, Cell Biochem Biophys 32: 187-204; Kliewer et al., 2001, Recent Prog Horm Res 56: 239-263). Three distinct PPARs, designated PPARα, PPARγ and PPARβ/δ, have been described in the scientific literature. In addition, PPARγ consists of two variants, PPARγ1 and PPARγ2. Each PPAR is differentially expressed in many tissues, and location is closely related to function.

PPARs are primarily responsible for lipid metabolism and homeostasis. They control the expression of genes associated with lipid metabolism through a sequence-specific promoter element termed the PPRE (AGGTCAGCTGTCA). While the endogenous PPAR ligands have not been identified, the PPARs can be activated by a diverse group of lipid or lipid-like compounds such as long chain fatty acids, eicosanoids and synthetic peroxisome proliferators. In addition to their structural differences, these agonists are also classified by their ability to selectively activate either PPARα or PPARγ, or to non-selectively activate both. It has been assumed the PPARs serve as targets for omega-3 fatty acids and thus promote a reduction in fat storage, and consequently obesity. Evidence to support this premise is reinforced by the established roles of PPARγ in adipocyte differentiation and PPARα in the regulation of fatty acid oxidation.

Based on this information, PPARα agonists have been proposed for the treatment of obesity (U.S. Pat. No. 6,028,109). Additionally, the scientific literature teaches that PPARα agonists decrease the inflammation and lipid deposition associated with atherosclerosis (Neve et al., 2000, Biochem Pharmacol 60: 1245-1250; Elangbam et al., 2001, Toxicol Pathol 29: 224-231). No claim has been made, however, that PPARα agonists operate as either anti-proliferative or anti-inflammatory agents. In fact, it has been reported that PPARα is not expressed in rat aortic smooth muscle cells (SMCs), although it has been detected in SMCs derived from rat mesenteric arteries and human saphenous vein (Diep et al., 2000, Hypertension 36: 851-855; Marx et al., 1998, Circ Res 83: 1097-1103). Similarly, there is no evidence to indicate that PPARα agonists influence smooth muscle cell proliferation, a major component of atherosclerosis. Conversely, agonists of PPARγ have been shown to prevent the proliferation of smooth muscle cells and keratinocytes (Hsuch et al., 2001, Diabetes Care 24: 392-397; Ellis et al., 2000, Arch Dermatol 136: 609-616). Consequently, PPARγ agonists have been proposed as treatments for restenosis after angioplasty and for proliferative skin diseases (U.S. Pat. Nos. 6,034,110 and 5,981,586).

PCT Patent Application WO 00/30628 teaches the inhibition of angiogenesis and tumor growth using PPARγ ligand/agonists. It is of note that while this document states that “PPARα ligands which, in addition to binding PPARα, also bind PPARγ (referred to as PPAR α/γ ligands)” may also be used, it further states that “it is generally necessary to administer the PPAR α/γ ligands in higher doses to achieve the same level of inhibition of angiogenesis as is obtained when a more specific PPARγ ligand is used”.

U.S. Pat. No. 6,214,850 teaches a class of PPARγ modulators and describes their use for treating diabetes and obesity.

### SUMMARY OF THE INVENTION

According to a first aspect of the invention, there is provided a pharmaceutical composition comprising a PPARα agonist; and a suitable excipient.

According to a second aspect of the invention, there is provided a method of treating or preventing a PPARα-related disease comprising:
administering an effective amount of a pharmaceutical composition comprising a PPARα agonist and a suitable excipient to an individual in need of such treatment.

According to a third aspect of the invention, there is provided a kit comprising a PPARα agonist for treating or preventing a PPARα-related disorder and instructions for administration of said PPARα agonist for the treatment of an injury-related disorder.

### BRIEF DESCRIPTION OF THE DRAWINGS

FIG. 1—Porcine and human smooth muscle cells express both PPARα and PPARγ1 mRNA. Porcine coronary artery smooth muscle cells (SMCs) were prepared by explant culture. Human saphenous vein, human internal mammary artery and human radial artery SMCs were prepared by a modification of the explant culture system. All cells were propagated in Dulbecco's modified Eagles media containing 20% FBS. The SMCs were prepared on 6-well culture dishes, and total cell RNA was extracted with TRIzol. The RNA (1 μg) was amplified by reverse-transcriptase polymerase chain reaction (RT-PCR). Amplification products were examined by ethidium bromide staining after agarose gel electrophoresis. Primers were designed using sequence information deposited in the Genbank database, and specificity was verified by sequencing the subcloned amplification products. The figure illustrates representative data for porcine coronary artery SMCs (Panel A), and human saphenous vein (Saph), human radial artery (Rad) and human internal mammary artery (IMA) SMCs (panel B) for the various PPAR isoforms as indicated.

FIG. 2—WY-14643 inhibits DNA synthesis by porcine coronary artery, human saphenous vein and human internal mammary artery SMCs in response to platelet-derived growth factor (PDGF) stimulation. SMCs were prepared by explant culture as previously described, grown in 24-well culture dishes to 70% confluence and then placed into serum-free DMEM supplemented with insulin, ascorbate, pyruvate and selenium for 5 days. These conditions result in the cessation of cell growth, and markers of the contractile state are expressed. The cells were subsequently treated with PDGF (0.1 μg) in the presence or absence of WY-14643, and 2 μCi/mL [³H]thymidine were added to the culture medium 24 hours later. Seventy-two hours following PDGF treatment, incorporation of thymidine into trichloroacetate-precipitable material was measured. The data, which is presented as means±s.e. (n=3), are shown for porcine SMCs (panel A), human saphenous vein SMCs (panel B) and human internal mammary artery SMCs (panel C). All values have been normalized relative to the incorporation in unstimulated quiescent cells (control=100).

FIG. 3—PGJ₂ inhibits DNA synthesis by porcine coronary smooth muscle cells following mitogenic stimulation with PDGF. Quiescent SMCs were treated with PDGF (01. μg/ml) in the presence and absence of PGJ₂, and incorporation of thymidine was measured as previously described. The data are presented as means±s.e. (n=3).

FIG. 4—WY-14643 does not inhibit DNA synthesis by A10 smooth muscle cells in response to either PDGF or insulin-like growth factor-1 (IGF-1) stimulation. A1-SMCs were cultured and quiescent cells were prepared in 24-well culture dishes as described in the legend for FIG. 2. The cells were treated with PDGF (0.1 μg) or IGF-1 (0.1 μM) in the presence or absence of WY-14643, and 2 μCi/mL [³H]thymidine were added to the medium concurrent with the mitogens. Seventy-two hours following treatment, the cells were lysed and thymidine incorporation was measured as described above. The figure shows the data, which is presented as means±s.e. (n=3), for A10 cells treated with PDGF (panel A) and IGF-1 (panel B).

FIG. 5—A10 SMCs do not express PPARα mRNA. A10 cells were prepared in 6-well dishes and total RNA was extracted. RT-PCR of total cellular RNA for PPARα, PPARγ1, PPARγ2 and GAPDH (C) was conducted as described for FIG. 1.

FIG. 6—WY-14643 inhibits neointimal proliferation in an organ culture model of restenosis. Porcine coronary arteries were subjected to balloon angioplasty ex vivo and placed into culture for 14 days. Treatment with WY-14643 (250 μM) was continuous over the entire time period. Subsequently, the vessel segments were sectioned and morphometry was employed to quantify the extent of restenosis. The neointimal index is defined as: neointimal area/medial area. The data are presented as means±s.e. (n=8). Control=no balloon angioplasty.

FIG. 7—WY-14643 inhibits DNA synthesis by H4IIE hepatoma cells in response to stimulation by IGF-1. H4IIE hepatoma cells were cultured and quiescent cells were prepared in 24-well culture dishes by placing the cells into serum-free α-MEM for 3 days. The cells were treated with IGF-1 (0.1 μM) in the presence or absence of WY-14643, and 2 μCi/mL [³H]thymidine were added concurrent with the treatment. Seventy-two hours following treatment, the cells were lysed and thymidine incorporation was measured as described above. The data are presented as means+/−s.e. (n=3).

FIG. 8—WY-14643 inhibits the growth of human proximal tubule cells. Human proximal tubule cells were purchased from Clonetics and propagated in 24-well culture dishes with Renal Epithelial Cell Basal medium (REBM) containing 0.5% fetal bovine serum (FBS). Treatments were added once the cells reached 40% confluence. Incubation was continued for 4 days, at which time the cells were detached by trypsinization and cell number measured with a Coulter counter. All comparisons are relative to a cell population treated with 5 μg/ml aphidicolin, a non-toxic inhibitor of cell proliferation. The data are presented as means+/−s.e. (n=6).

FIG. 9—PGJ₂ inhibits basal DNA synthesis by porcine coronary artery smooth muscle cells and H4IIE hepatoma cells in the absence of mitogenic stimulation, but WY-14643 has no effect. Quiescent SMCs (panel A) and H4IIE cells (panel B) were treated with PGJ₂ and incorporation of thymidine was measured as previously described. Quiescent SMCs were also treated with WY-14643 (panel C) and incorporation of thymidine was measured. The data are presented as means±s.e. (n=3).

FIG. 10—Both H4IIE hepatoma and human proximal tubule cells express PPARα mRNA. Total cellular RNA was extracted from cells grown in 6 well culture dishes and RT-PCR was conducted as described for FIG. 1. This figure illustrates representative data for H4IIE hepatoma cells (panel A) and human proximal tubule cells (panel B). C=GAPDH.

FIG. 11—Rabbit SMCs express PPARα and PPARγ mRNA. Rabbit aortic SMCs were prepared by explant culture according to the methods already described. The cells were prepared in 6-well culture dishes and total RNA was extracted. RT-PCR of the RNA was conducted as described above. Γ=PPARγ1+PPARγ2 (primers to distinguish the individual isoforms could not be designed with the limited sequence available). C=GAPDH.

FIG. 12—Clofibrate inhibits DNA synthesis by porcine coronary SMCs in response to PDGF stimulation. Quiescent SMCs were treated with PDGF (0.1 μg) in the presence or absence of clofibrate, and incorporation of thymidine was measured as previously described. The data are presented as means±s.e. (n=3).

FIG. 13—Lack of inhibition by PPAR-gamma agonists on DNA synthesis by smooth muscle cells.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “PPARα agonist” refers to compounds which activate PPARα. Examples include but are by no means limited to WY-14643, clofibrate, benzafibrate, fenofibrate, GW409544 and BM-17.0744.

As used herein, “PPARα-related disease” includes restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids).

As used herein, “effective amount” refers to the administration of an amount of a given compound that achieves the desired effect.

As used herein and as discussed above, “vascular stenosis” refers to vessel wall thickening, clogging or constriction and loss of blood flow. The stresses leading to stenosis may be, for example, mechanical, hypoxia, injury, shear-stress, pharmacological, infectious, inflammatory, oxidative, immunogenic, diabetic or pressure.

As used herein and as discussed above, “angioplasty” refers to procedures and methods involved in the opening or unclogging of blocked arteries, including stents. In some instances, angioplasty involves the use of a balloon-tipped catheter which is inserted into the heart's vessels to open partially blocked, or stenotic, coronary arteries. While balloon angioplasty does widen the restricted artery, a significant number of patients have renewed narrowing of the widened segment soon after the procedure. This subsequent narrowing of the artery is called restenosis and can necessitate the repetition of the angioplasty procedure or require alternative treatment such as coronary bypass graft surgery.

“Bypass graft surgery” refers to procedures and methods involved in the treatment of atherosclerotic or restenotic lesions that restrict blood flow. In some instances, bypass grafting involves the joining of a vein to an artery to produce a conduit that allows blood to flow around a blockage. In other cases, the graft consists of two arteries. The junction of the vessels is termed the anastomosis and it is a region that can also narrow, that is, undergo restenosis.

It is of note that restenosis may be associated with or caused by any vascular injury, since it is the injury resulting from angioplasty, stenting or bypass grafting that leads to restenosis.

Herein, it is demonstrated that PPARα agonists prevent the proliferation of hepatic, renal and vascular cells. This anti-proliferative and anti-inflammatory activity can be employed in the treatment of restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids).

Specifically, it is shown that PPARα agonists inhibit cell proliferation. It is of note that an anti-proliferative role for PPARα has not been conclusively indicated nor has the involvement of PPARα in any pathogenic state been conclusively demonstrated. PPARα agonists are currently used to treat only a subset of atherosclerotic patients, namely, those with high triglyceride levels. Atherosclerotic patients without high triglyceride levels, but with high cholesterol levels are not treated with PPARα agonists. The present findings suggest that PPARα agonists have an anti-proliferative effect and would be effective in the treatment of all patients with atherosclerosis. Thus, rather than having a modulatory role in the progression of such diseases, PPARα agonists may be effective in the treatment of atherogenesis and other proliferative disease.

The PPARα agonists are useful as therapeutic agents to control diseases such as restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids). In some embodiments, the PPARα agonists are used in conjunction with a PPARγ agonist. In other embodiments, the PPARα agonists are used in conjunction with an anti-proliferative drug. In yet other embodiments, the PPARα agonists are used in combination with retinoids and other agonists of the RXR receptor. Although we focus primarily on WY-14643, data also indicates that other PPARα agonists (e.g. Clofibrate) can also inhibit cell proliferation (FIG. 12). Thus, the properties ascribed to WY-14643 are applicable to other PPARα activators, for example, clofibrate, benzafibrate, fenofibrate, GW409544 and BM-17.0744. Finally, it should be recognized that there are tremendous advantages in employing an endogenous inhibitor of cell proliferation such as a PPARα agonist which does not exhibit cytotoxicity (FIG. 9). This property is highly relevant given the limited treatment options for most anti-proliferative agents currently in clinical use as a result of their toxicity.

In some embodiments, the PPARα agonist is arranged to be administered at a concentration of 0.001%-0.1%, referring to grams of agonist per 100 grams of food. As will be appreciated by one of skill in the art, the actual dosage may vary according to patient condition and dosage necessary to elicit the desired response.

It is of note that the PPARα agonist discussed above may be prepared to be administered in a variety of ways, for example, topically, locally (delivered for example to a tissue), orally, intravenously, intramuscularly, subcutaneously, intraperitoneally, intranasally or by local or systemic intravascular infusion using means known in the art and as discussed below.

In some embodiments, the PPARα agonist may be combined with other compounds or compositions known in the art such that the PPARα agonist is in the form of, for example, an ointment, pill, tablet, cream, suppository, lotion, gel, foam, film, barrier, wrap, paste or coating using means known in the art and as discussed below. Preferably, such films, wraps or barriers are generally less than 5, 4, 3, 2 or 1 mm thick. In some embodiments, the film may be less than 0.75 mm or 0.5 mm thick. Preferably, the films have good tensile strength and good adhesive properties.

In some embodiments, the PPARα agonist at concentrations or dosages discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

In yet other embodiments, the PPARα agonist as dosages described above may be contained within or adapted to be released by a surgical or medical device, for example, stents, catheters, prostheses, sutures and the like. In these embodiments, the PPARα agonist at concentrations or dosages described above may be incorporated into nylon microcapsules and applied to the surface of the stent or device. Alternatively, the device may be coated with a film composed of, for example, cellulose, hyaluronic acid, chitosan, ethylene vinyl acetate, or poly lactic acid, impregnated with the PPARα agonist. Yet further, the device may be coated with a thermo-sensitive gel such that the PPARα agonist is released when the device is implanted.

Typically, stents are used to expand the lumen of a body passageway. This involves inserting the stent into the passageway such that the passageway is expanded. In general, a preinsertion examination, for example, either a diagnostic imaging procedure or direct visualization at the time of surgery is performed to determine the appropriate location for stent insertion. First, a guide wire is advanced through the proposed site of insertion. A delivery catheter is then passed over the guide wire, allowing insertion of the catheter into the desired position. The stent is then expanded by means known in the art.

The stent may be coated for example by spraying or dipping the stent with or in the PPARα agonist described above, or the stent may be coated with an absorption-promoting substance, such as hydrogel, first. Alternatively, the stent may be surrounded in a sleeve, mesh or other structure impregnated with the PPARα agonist and arranged to release the PPARα agonist over time.

In other embodiments, a PPARα agonist at concentrations or dosages described above may be encapsulated for delivery. Specifically, the PPARα agonist may be encapsulated in biodegradable microspheres, microcapsules, microparticles, or nanospheres. The delivery vehicles may be composed of, for example, hyaluronic acid, polyethylene glycol, poly(lactic acid), gelatin, poly(E-caprolactone), or a poly(lactic-glycolic) acid polymer. Combinations may also be used, as, for example, gelatin nanospheres may be coated with a polymer of poly(lactic-glycolic) acid. As will be apparent to one knowledgeable in the art, these and other suitable delivery vehicles may be prepared according to protocols known in the art and utilized for delivery of the PPARα agonist. In some embodiments, the delivery vehicle may be coated with an adhesive for localizing the PPARα agonist to the area of interest. Alternatively, the delivery vehicle may be suspended in saline and used as a nanospray for aerosol dispersion onto an area of interest. Furthermore, the delivery vehicle may be dispersed in a gel or paste, thereby forming a nanopaste for coating a tissue or tissue portion.

It is of note that the PPARα agonist as described above may be combined with permeation enhancers known in the art for improving delivery. Examples of permeation enhancers include, but are by no means limited to those compounds described in U.S. Pat. Nos. 3,472,931; 3,527,864; 3,896,238; 3,903,256; 3,952,099; 4,046,886; 4,130,643; 4,130,667; 4,299,826; 4,335,115; 4,343,798; 4,379,454; 4,405,616; 4,746,515; 4,788,062; 4,820,720; 4,863,738; 4,863,970; and 5,378,730; British Pat. No. 1,011,949; and Idson, “1975, J. Pharm. Sci. 64:901-924.

In some embodiments, the PPARα agonist in any suitable form as described above, may be combined with biological or synthetic targetting molecules, for example, site-specific binding proteins, antibodies, lectins or ligands, for targetting the PPARα agonist to a specific region or location.

The invention provides kits for carrying out the methods of the invention. Accordingly, a variety of kits are provided. The kits may be used for any one or more of the following (and, accordingly, may contain instructions for any one or more of the following uses): treating restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids) in an individual, preventing vascular constriction, swelling, pain, inflammation, or rapid cell or tissue growth in an individual at risk of restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids); preventing one or more symptoms of vascular constriction, swelling, pain, inflammation or rapid cell or tissue growth or the like in an individual at risk of restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids); reducing severity of one or more symptoms of vascular constriction, swelling, pain, inflammation, or rapid cell or tissue growth in an individual; reducing recurrence of one or more symptoms of vascular constriction, swelling, pain, inflammation, or rapid cell or tissue growth in an individual; suppressing vascular constriction, swelling, pain, inflammation or rapid cell or tissue growth in an individual at risk of restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids); delaying development of vascular constriction, swelling, pain, inflammation or rapid cell or tissue growth and/or a symptom of restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids) in an individual; reducing duration of vascular constriction, swelling, pain, inflammation, or rapid cell or tissue growth in an individual.

The kits of the invention comprise one or more containers comprising a PPARα agonist, a suitable excipient as described herein and a set of instructions, generally written instructions although electronic storage media (e.g., magnetic diskette or optical disk) containing instructions are also acceptable, relating to the use and dosage of the PPARα agonist for the intended treatment (e.g., restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids)). The instructions included with the kit generally include information as to dosage, dosing schedule, and route of administration for the intended treatment. The containers of the PPARα agonist may be unit doses, bulk packages (e.g., multi-dose packages) or sub-unit doses.

The PPARα agonist of the kit may be packaged in any convenient, appropriate packaging. For example, if the PPARα agonist is a freeze-dried formulation, an ampoule with a resilient stopper is normally used, so that the drug may be easily reconstituted by injecting fluid through the resilient stopper. Ampoules with non-resilient, removable closures (e.g., sealed glass) or resilient stoppers are most conveniently used for injectable forms of the PPARα agonist. Also, pre-filled syringes may be used when the kit is supplied with a liquid formulation of the PPARα agonist. The kit may contain the PPARα agonist in an ointment for topical formulation in appropriate packaging.

Atherosclerosis is the development of arterial lesions in the coronary arteries that feed the heart (coronary artery disease) and cause heart attacks and heart failure. These lesions are typically due to elevated cholesterol and triglycerides levels in the blood, in combination with proliferation of vascular tissue. Lesions affect blood flow to the heart, as well as to other organs and the extremities, resulting in for instance reduced kidney function and stroke. As atherosclerosis is accelerated in diabetics, the incidence of limb amputation, retinopathy and neuropathy seen in these patients provide further examples of the effects of arterial function on hospitalization rates. Treatment of early atherosclerosis is primarily accomplished by altering life style. Subsequently, lipid-lowering agents are the preferred medicaments, as there is currently no available method of inhibiting the vascular proliferation that contributes to atherosclerosis. The statins (HMG-CoA reductase inhibitors) are primarily indicated in the treatment of atherosclerosis in patients with elevated cholesterol, however, Baycol was recently withdrawn from the market. Conversely, in patients with high triglyceride levels, fibrates (e.g. clofibrate) are the drugs of choice. PPARα agonists will inhibit the proliferation associated with the pathogenesis of atherosclerosis. Furthermore, all patients with high triglycerides or high cholesterol would benefit from PPARα agonist treatment.

Restenosis remains an unresolved medical issue and any progress in reducing the occurrence of arterial re-narrowing would be revolutionary. The most relevant hospital procedures that result in restenosis are balloon angioplasty and bypass graft surgery. This occurs, but not exclusively, when the stent from angioplasty is reclogged with scar tissue. Coronary artery bypass grafts are the most prevalent, and there are 500,000 procedures annually in the United States. In this procedure, restenosis occurs early (2 weeks-6 months) at the site of the graft (anastomosis). Late failure (2-10 years) occurs as a result of atherosclerosis in the grafted vessel, limiting graft lifespan to approximately 10 years. An added benefit of PPARα agonist treatment for patients undergoing bypass grafting is that both early failure due to restenosis and late failure due to atherosclerosis may be prevented. Unlike stents, there is no treatment available to prevent restenosis in these patients. Furthermore, it is known that there are inflammatory and proliferative components that contribute to the development of an arteriosclerotic lesion. Thus, by inhibiting expression of pro-inflammatory genes and proliferation of vascular cells, a PPARα agonist would restrict progression of this condition. Furthermore, all vascular procedures involving grafting, puncturing or producing intimal damage can be treated by the above-described compounds, as could valve replacements, catheters, prosthesis, implanted devices, pacemakers, nerve stimulators, patches, organ transplants, small vessel vasculopathy, wound repair, psoriasis, as well as any inflammatory or proliferative disease that is localized to a defined region. In these embodiments, the PPARα agonist may be localized through the use of an adhesive, impregnated mesh or targetting molecule as described herein, or the device or organ may be coated or infused with the PPARα agonist as described herein.

An estimated 5.6 million Americans have elevated serum creatinine levels in their blood, which is an indicator of chronic kidney disease. The incidence of chronic kidney disease is rising by 7-8% annually. This population is at greater risk of developing kidney failure, anemia, bone disease, ischemic heart disease and death. Better management of hypertension has led to fewer strokes and heart disease, but kidney failure continues to increase. Many patients with chronic renal failure do not have symptoms until their kidney function has decreased to less than 25 percent of normal. Chronic kidney failure (sometimes described as End Stage Renal Disease—ESRD) is currently the sixth leading cause of death in Canada. Diabetes is the most common cause of chronic renal failure, followed by hypertension. This will exponentially increase the incidence of renal failure in the future as type 2 diabetes becomes ever more prevalent in the general population. As discussed above, the PPARα agonists inhibit renal cell proliferation, meaning that treating individuals suffering from diseases that may lead to chronic kidney failure, for example, diabetes, will prevent development of chronic kidney failure. It is also of note that diabetes is a risk factor for cardiovascular disease and the increasing incidence of diabetes will lead to more cardiovascular disease complications.

Benign hyperplastic prostate or nodular hyperplasia of the prostate is initiated by a proliferation of the mesenchymal-stromal or the glandular-epithelial portion of the gland. Like prostate cancer, benign hyperplastic prostate is characterized by a need to urinate frequently, especially at night; difficulty starting urination or holding back urine; inability to urinate; weak or interrupted flow of urine; painful or burning urination; painful ejaculation; blood in urine or semen; and/or frequent pain or stiffness in the lower back, hips, or upper thighs. Administering PPARα agonists to individuals suffering from these diseases will at least alleviate these symptoms.

As discussed above, arthritis is believed to be an autoimmune disease, characterized by infiltration of the joints with inflammatory system cells. As discussed above, PPARα agonists inhibit expression of proliferative and inflammatory genes, indicating that these compounds would be an effective treatment for arthritis. In these embodiments, the PPARα agonist is arranged to be injected directly into the afflicted joints or taken orally. Preparation of the PPARα agonist for injection is described herein.

As discussed above, asthma is characterized by recurring airway obstruction involving smooth muscle cell proliferation and inflammatory cell infiltration. Given that the PPARα agonists inhibit expression of proliferative and inflammatory genes, these compounds would likely lessen the severity of asthma attacks. That is, the PPARα agonist would accomplish one or more of the following: decrease the severity of or ameliorate symptoms, decrease the duration of attacks, increase the frequency and duration of remission periods, prevent chronic progression of dyspnea, coughing and wheezing, improve hypoxia, increase forced expiration volume in one second, and improve resistance to airflow and hypocapnea/respiratory alkalosis. In embodiments for treating asthma, the PPARα agonist may be arranged to be inhaled, for example, in a spray form, the preparation of which is described herein.

The mechanism of fibrotic disease or fibrosis is still not fully understood, but wound healing usually begins as an inflammatory reaction with leucocyte infiltration and accumulation of cytokines. These cytokines are responsible for the proliferation of fibroblasts and the deposition of extracellular matrix proteins (including collagen and fibronectin) which accumulate and result in permanent alteration in tissue structure and function. As discussed above, given that the PPARα agonists inhibit expression of proliferative genes, these compounds would be an effective treatment for any fibrotic disease.

Similarly, inflammatory bowel diseases are caused by intestinal inflammation and repeated inflammatory responses. As discussed above, the PPARα agonists inhibit expression of pro-inflammatory genes, meaning that the PPARα agonists would also be an effective treatment for these disorders. That is, injection or infusion of the PPARα agonists into the bowel or intestine will inhibit migration of cells of the inflammatory system, thereby reducing the severity of the disease. Specifically, the PPARα agonists would accomplish at least one of the following: decrease the frequency of the attacks, increase the duration of remission periods, decrease the severity or duration of abscess formation, intestinal obstruction, intestinal perforation and the like as well as ameliorate or reduce symptoms such as bloody diarrhea, abdominal pain, fever, weight loss and abdominal distension. Examples of inflammatory bowel diseases include but are by no means limited to Crohn's disease and ulcerative colitis.

The following Observations are provided to illustrate, but not limit, the invention.

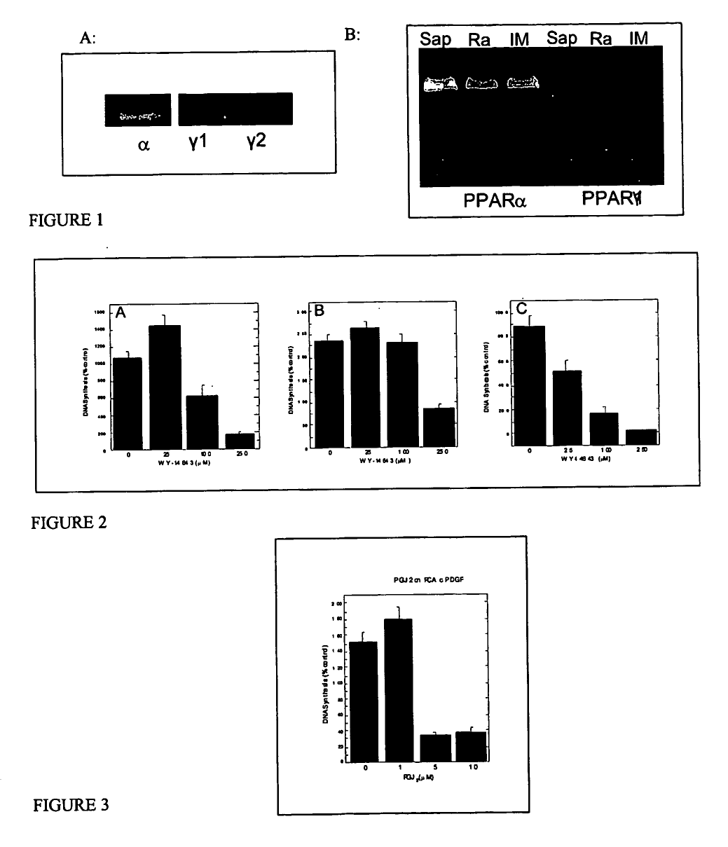

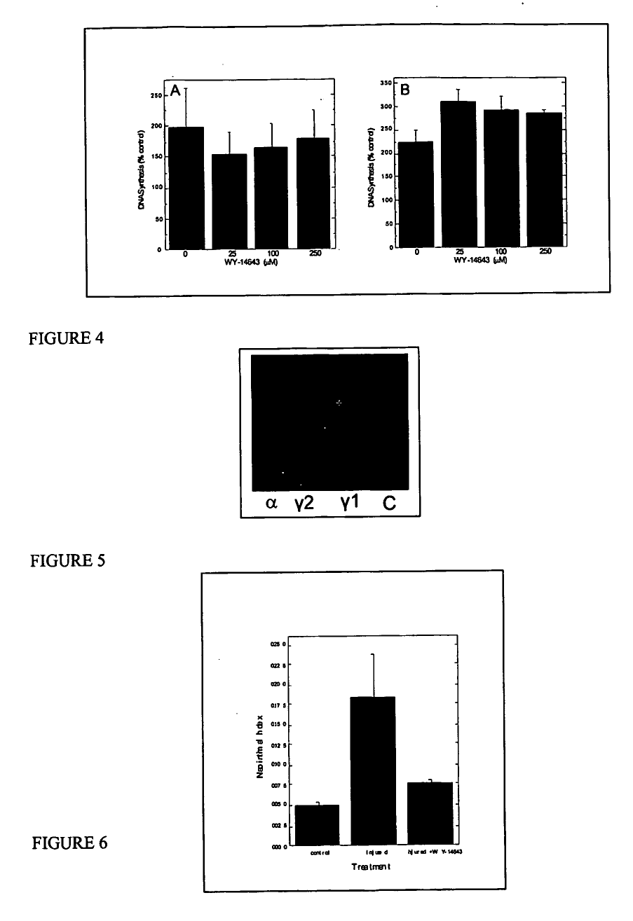

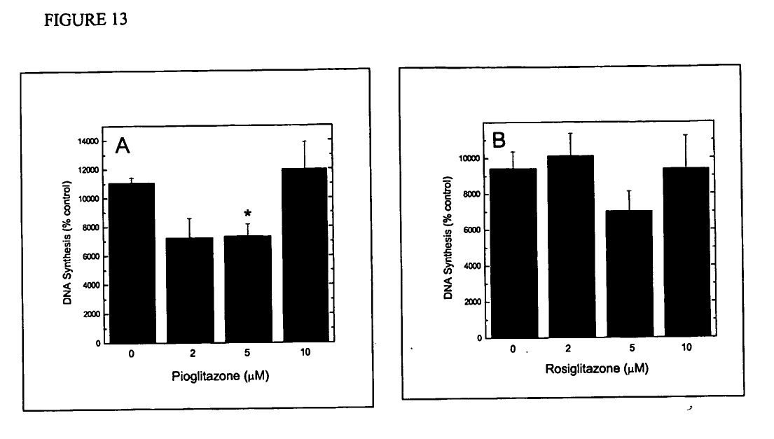

Investigation of PPARs as mediators of the benefits obtained with nutritional supplementation led to an examination of PPAR expression in liver, kidney, adipose, heart and vasculature tissue. Reverse transcriptase polymerase chain reaction (RT-PCR) amplification of mRNA isolated directly from these tissues, as well as from cells derived from these same tissues, revealed that PPARα was expressed by smooth muscle cells of porcine coronary artery, human saphenous vein and human internal mammary artery, as shown in FIG. 1. The ability of various PPAR agonists to block smooth muscle cell growth was then tested, assuming that PPARγ agonists would be effective (Hsuch et al., 2001, Diabetes Care 24: 392-397). Surprisingly, WY-14643, a strong PPARα agonist, inhibited DNA synthesis (FIG. 2) as effectively as the PPARγ agonist PGJ₂ (FIG. 3) by SMCs from 3 distinct sources. A link with PPARα was strengthened by our finding that WY-14643 could not inhibit the growth of rat A10 smooth muscle cells (FIG. 3), a cell line that does not express PPARα (FIG. 4). Thus, the common presumption that PPARα has no role in smooth muscle cell growth (based on studies of rat tissues) is erroneous. In fact, more detailed studies of PPARγ agonists have shown that the inhibitory effect of PGJ₂ occurs independent of any effect on PPARγ. This observation is supported by data, which shows PGJ₂ is inhibitory (FIG. 3) whereas the selective PPARγ agonists rosiglitazone and piaglitazone are not (FIG. 13). Given this information, we have postulated that PPARα agonists could be employed clinically to control the proliferation of vascular smooth muscle cells. This premise was confirmed by showing WY-14643 prevents restenosis in a porcine organ culture model of balloon angioplasty (FIG. 6). Furthermore, a recent publication has confirmed the observation that PPARα agonists are capable of blocking proliferation of smooth muscle cells (Nigro et al., 2002, Atherosclerosis 162: 119-129).

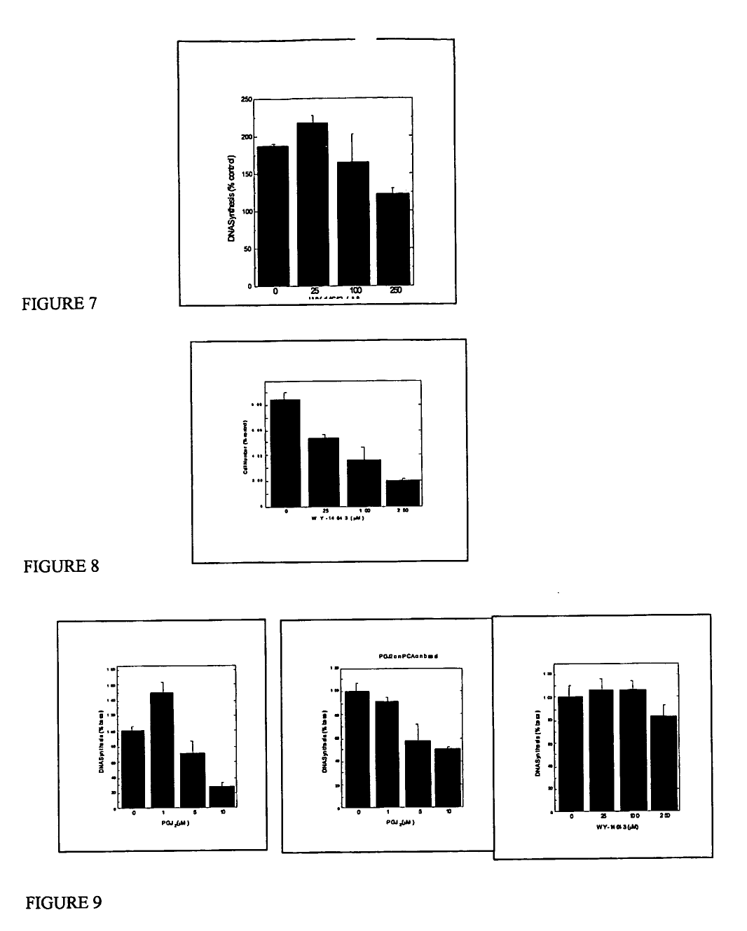

Expression of PPARα in kidney and liver has been reported (Braissant et al., 1996, Endocrinology 137: 354-366). Nevertheless, there is no published report examining the effect of PPARα agonists on the proliferation of cells from these tissues. Given our observation with smooth muscle cells, WY-14643 was tested with both hepatoma and primary cultures of human renal proximal tubular cells. In both cases, WY-14643 prevented cell growth (FIGS. 7,8) without apparent toxicity (FIG. 9). These data confirm and extend the initial observation and suggest that activation of PPARα results in an inhibition of cell proliferation.

The following represent example applications for PPARα agonists:

### EXAMPLE I

### Treatment of Atherosclerosis

High dietary levels of either cholesterol or fat (specifically triglycerides) promote the formation of lipid-filled arterial lesions. Both cholesterol and lipid lowering agents have therefore been used clinically to limit the development of atherosclerosis. It is interesting that the most successful agents are the statins, inhibitors of HMG-CoA reductase, an enzyme required for cholesterol synthesis. However, there is convincing evidence that statins inhibit cellular proliferation by a mechanism that is independent of their effect on cholesterol levels (Bellosta et al., 1998, Atherosclerosis 137 Suppl: S101-109). In contrast, the fibrates, a distinct family of lipid-lowering agents, operate via PPARα to increase hepatic β-oxidation of fatty acids (Watts et al., 1999, Curr Opin Lipidol 10: 561-574; Gervois et al., 2000, Clin Chem Lab Med 38: 3-11), thus decreasing triglyceride synthesis and lowering serum lipid levels. These compounds, therefore, are suitable for the treatment of primary hypertriglyceridemia and mixed hyperlipidemia. However, the minute decrease in serum LDL levels provided by fibrates makes them of limited use for the treatment of hypercholesterolemia (Fruchart et al., 1998, Am J Cardiol 81: 912-917). Dietary treatment with WY-14643 will involve supplementation with 0.001% WY-14643 to 0.01% WY-14643, expecting an amount of 0.0025% to show efficacy. The method of delivery could be via a capsule or by inclusion in specific food materials. A decrease in the area of coronary vessels covered by atheromatous lesions should be present. The ability to reduce atherosclerosis should be independent of the lipid lowering effect as determined by measuring fasting serum cholesterol (total, LDL and HDL) and triglyceride levels.

### EXAMPLE II

### Treatment of Restenosis

The development of vascular lesions as a result of a revascularization procedure (typically a 40% incidence within 6-12 months) is coupled to an increase in the proliferative capacity of SMCs (Rivard et al., 2000, Histol Histopath 15: 557-571). Therapeutic agents capable of inhibiting smooth muscle cell proliferation thus have the ability to prevent lesion formation. Agents demonstrated to reduce the rate of restenosis post-angioplasty include chemotherapeutic compounds such as paclitaxel (taxol), colchicine and cytochalasin B (Hong et al., 2001, Coron Artery Dis 12: 513-515; Gradus-Pizlo et al., 1995, J Am Coll Cardiol 26: 1549-1557; Lehmann et al., 2000, J Am Coll Cardiol 35: 583-591). Anti-inflammatory agents (eg. tranilast, probucol) also have been observed to reduce restenosis rates (Ishiwata et al., 2000, J Am Coll Cardiol 35: 1331-1337; Rodes et al., 1998, Circulation 97: 429-436). In contrast, lipid-lowering agents such as statins are ineffective (Horlitz et al., 2001, Herz 26: 119-128). Our data reveals that activation of PPARα by WY-14643, employing a supplementation as described in Example I, can inhibit smooth muscle cell proliferation and prevent restenosis (FIGS. 2A, 4), as defined by standard clinical practice.

### EXAMPLE III

### Treatment of Chronic Renal Failure

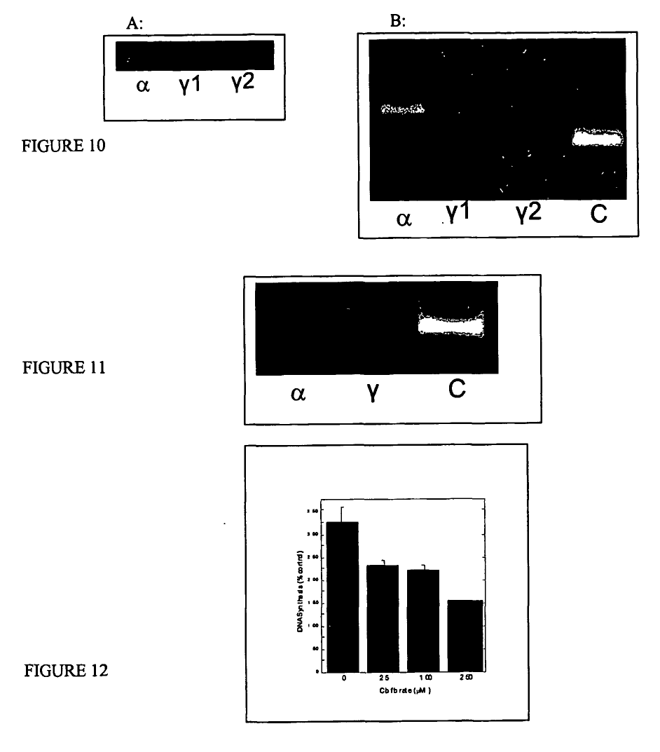

Chronic kidney failure progresses to end stage kidney disease as a result of glomerular capillary hypertension and kidney cellular hypertrophy (Fogo, 2000, Kidney Int Suppl 75: S15-21). The role of kidney hypertrophy in the progression of chronic renal failure is highlighted by the finding that only diabetics with enlarged kidneys go on to develop kidney failure. Furthermore, individuals with other forms of kidney disease have a much higher chance of developing progressive kidney failure if there is compartmental hypertrophy within their kidneys (Wolf and Ziyadeh, 1999, Kidney Int 56: 393-405; Fogo et al., 1990, Kidney Int 38: 115-123). Provided that they are started at an early stage, angiotensin converting enzyme inhibitors and angiotensin II receptor blockers, can delay (by <6 months), but not halt the progression, of diabetic kidney disease (Myers et al., 1998, J Am Soc Nephrol 9: S66-S70). These agents operate by decreasing glomerular capillary pressure, but cannot prevent the compensatory cellular hypertrophy that accompanies most forms of kidney disease. We and others have demonstrated that human proximal tubular cells express PPARα mRNA (FIG. 7). WY-14643 effectively inhibits the growth of primary cultures of human renal proximal tubule cells (these cells comprise 70% of the cellular mass of the kidney) in response to IGF-1 without apparent toxicity (FIG. 10). These data indicate PPARα agonists such as WY-14643, delivered by methods as described in Example I, have potential as a renal salvage therapy for human chronic renal failure. Both renal function and renal blood flow as determined by inulin and para-aminohippurate clearance studies, respectively, would be expected to show improvement relative to untreated individuals.

While the preferred embodiments of the invention have been described above, it will be recognized and understood that various modifications may be made therein, and the appended claims are intended to cover all such modifications which may fall within the spirit and scope of the invention.

1. A pharmaceutical composition comprising a PPARα agonist; and a suitable excipient.

2. The pharmaceutical composition according to claim 1 wherein the PPARα agonist is selected from the group consisting of WY-14643, clofibrate, benzafibrate, fenofibrate, GW409544 and BM-17.0744.

3. A method of treating or preventing a PPARα-related disease comprising:
administering an effective amount of a pharmaceutical composition comprising a PPARα agonist and a suitable excipient to an individual in need of such treatment.

4. The method according to claim 3 wherein the PPARα-related disease is selected from the group comprising: restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids).

5. A kit comprising a PPARα agonist for treating or preventing a PPARα-related disorder and instructions for administration of said PPARα agonist for the treatment of an injury-related disorder.

6. The kit according to claim 5 wherein the PPARα-related disease is selected from the group comprising: restenosis, chronic renal failure, atherosclerosis, hypertension, diabetes, benign hyperplastic prostate, prostate cancer, rheumatoid arthritis, asthma, stress-induced inflammation, hypoxia, fibrotic disease, skin wound healing, keloids, Crohn's disease, ulcerative colitis, endometeriosis, fibromatosis, pulmonary hypertension, cardiac hypertrophy, congestive heart failure and leiomynomas (fibroids).
